(19) United States
(12) Patent Application Publication
(10) Pub. No.: US 2003/0148365 A1
(43) Pub. Date: Aug. 7, 2003

(54) Method of self-sampling and analyzing a semen sample

(76) Inventor: Allen Morgan, Brick, NJ (US)

Correspondence Address: EZRA SUTTON, PA, PLAZA 9, 900 ROUTE 9, WOODBRIDGE, NJ, 07095, US

(21) Appl. No.: 10/364,194

(22) Filed: Feb. 12, 2003

Related U.S. Application Data
(63) Continuation of application No. 09/766,041, filed on Jan. 19, 2001, now abandoned.

(51) Int. Cl. G06F 17/60; C12Q 1/68; A01N 1/02; C12Q 1/00

(52) U.S. Cl. 435/6; 435/2; 705/3

(57) ABSTRACT

A method of self-sampling and analyzing a semen sample without visiting a doctor's office, utilizing a kit including an insulated box containing an ice brick, a sample bottle, and a preservative solution, including the steps of:

1) Ordering a kit by telephone or on the Internet;

2) Receiving and placing the kit in a freezer until ready for use;

3) Placing a semen sample into the sample bottle and adding the preservative solution to the sample bottle;

4) Placing the sample bottle into the kit next to the ice brick in the insulated box;

5) Shipping the kit and the sample bottle to a diagnostic laboratory for analysis of the semen sample;

6) Analyzing the semen sample on a semen analyzer at the diagnostic laboratory;

7) Transmitting a confidential analysis report to the patient setting forth analysis of the semen sample;

8) Consultation service for the patient; and

9) Referral service for the patient.

### RELATED APPLICATION

This is a continuation application of the parent patent application Ser. No. 09/766,041 filed on Jan. 19, 2001.

### FIELD OF THE INVENTION

This invention relates to a method of self-sampling and analyzing a semen sample. More particularly, this at-home semen analysis kit is an easy method in which to have access to an infertility medical laboratory without the user actually having to go to the laboratory in person. The at-home semen analysis kit provides the user the necessary equipment, preservative chemicals, paraphernalia, shipping boxes (return mailer) and instructions for the self-sampling of the user's semen at the privacy of the user's home, whereby the user will receive his results by mail within 48 hours of the semen sample being analyzed at the infertility medical laboratory.

### BACKGROUND OF THE INVENTION

Medical statistics have shown that in couples unable to conceive, approximately one-half of the time a male factor is involved. The sole cause is with the male alone 40% of the time, and 10% being combined male and female causes. Fifty percent of the time the factors can be attributed to the female partner only.

In addition, males with a known history of sexually transmitted disease, history of exposure to chemical, environmental, or other workplace hazards, may have an increased risk of an abnormal semen analysis.

Other factors associated with an abnormal semen analysis are: a history of surgery for undescended testes and inguinal hernias, excessive exposure to hot tubs and prolonged use of tight briefs. Anabolic steroids are definitely a cause of low sperm counts. Trauma to the groin area also my be a factor.

There remains a need for an easy way to have access to an infertility medical practice without actually having to go there in person by the user. Medical research has shown that sperm can survive for days in a preservative solution, this analysis can be accomplished without wasting hours going to and from a medical infertility laboratory as you can get the same information through the use of an overnight at-home semen and collection kit.

### DESCRIPTION OF THE PRIOR ART

Methods for transporting medical samples, such as semen, blood, bone marrow, skin tissue and the like by conventional overnight delivery/mail services have been disclosed in the prior art. For example, U.S. Pat. No. 5,983,661 to Wiesman discloses a container arrangement and method for transporting equine semen. This container allows for transporting samples of equine semen over long distance while at the same time maintaining the motility and fertility of the transported spermatorozoa for at least 48 hours without the sperm being inactivated. This prior art patent does not teach or disclose a method of self-sampling and collection for analysis of a semen sample being an at-home semen collection and analysis kit of the present invention.

U.S. Pat. No. 6,018,713 to Coli et al discloses an integrated system and method for ordering and retrieving the cumulative results/reporting of medical tests. Further, this invention relates to an improved system and method for on-line ordering of medical tests in a health care network, an improved system and method for uniformly recording and reporting test results, and an improved system and method for collecting and statistically analyzing test results. This prior art patent does not teach or disclose a method of self-sampling and collection for analysis of a semen sample being an at-home semen collection and analysis kit of the present invention.

None of the prior art patents disclose or teach a method for self-sampling and collection of human semen sample for analysis through the use of an at-home semen collection and analysis kit as shown in the present invention.

Accordingly, it is an object of the present invention to provide an at-home semen collection and analysis kit that gives the user an easy way in which to have access to a medical infertility laboratory without the user actually having to go to the laboratory in person.

Another object of the present invention is to provide an at-home semen collection and analysis kit that includes the necessary collection equipment, the preservative chemicals, an overnite return mailer and box, and instructions for the self-sampling of the user's semen at the privacy of the user's home, whereby the user will receive his confidential report by mail within 48 hours of the semen sample being analyzed at the infertility medical laboratory.

Another object of the present invention is to provide an at-home semen collection and analysis kit, such that the user is not examined in a cold and impersonal medical facility, as well as having the mental anguish of sharing the user's most intimate medical information of his problems with an unknown medical staff.

Another object of the present invention is to provide an at-home semen collection and analysis kit that is easy to use, durable in construction, and having simple packing instructions for shipment to the medical infertility laboratory.

Another object of the present invention is to provide an at-home semen collection and analysis kit that maintains the integrity of the semen sample taken by the user prior to its analysis at the medical infertility laboratory, such that the sperm is in a viable state for proper analysis.

Another object of the present invention is to provide an at-home semen collection and analysis kit that enables the economic transport of human semen, in a disposable, totally self-contained, passively cooled, inexpensive container that has essentially no moving parts, and that is not equipped with thermostatically controlled cooling arrangements or the like, for periods of time approximating at least 24 to 48 hours.

A further object of the present invention is to provide an at-home semen collection and analysis kit that can be mass produced in an automated and economical manner and is readily affordable by the user.

### SUMMARY OF THE INVENTION

In accordance with the present invention, there is provided a method of self-sampling and analyzing a semen sample without visiting a doctor's office, utilizing a kit including a insulated box containing an ice brick, a sample bottle, and a preservative solution, including the steps of:

1) Ordering a kit by telephone or on the Internet;

2) Receiving and placing the kit in a freezer until ready for use;

3) Placing a semen sample into the sample bottle and adding the preservative solution to the sample bottle;

4) Placing the sample bottle into the kit next to the ice brick in the insulated box;

5) Shipping the kit and the sample bottle to a diagnostic laboratory for analysis of the semen sample;

6) Analyzing the semen sample on a semen analyzer at the diagnostic laboratory;

7) Transmitting a confidential analysis report to the patient setting forth analysis of the semen sample;

8) Allowing for a free consultation to discuss the results and referral to a specialist in their area if needed; and

9) Establishing a referral system.

### BRIEF DESCRIPTION OF THE DRAWINGS

Further objects, features, and advantages of the present invention will become apparent upon the consideration of the following detailed description of the presently-preferred embodiment when taken in conjunction with the accompanying drawings, wherein:

FIG. 1 is a schematic block diagram of the method of self-sampling and analyzing a semen sample of the preferred embodiment of the present invention showing all of the steps involved for the sampling, the shipping, the analysis and the reporting of results of a semen sample by the user's use of an at-home semen collection and analysis kit;

FIG. 2 is a front perspective view of the at-home semen collection and analysis kit of the preferred embodiment of the present invention showing the major component parts within the kit;

FIG. 3 is a top plan view of the at-home semen collection and analysis kit of the present invention showing the styrofoam retaining member, the foam insert having compartments therein, the semen collection bottle, the preservative chemical solution bottle and the ice brick;

FIG. 4 is an exploded perspective view of the at-home semen collection and analysis kit of the present invention showing all of the component parts being used for the kit; and

FIG. 5 is an exploded perspective view of the at-home semen collection and analysis kit of the present invention showing the kit, the outer shipping container and the overnite express lab pack envelope being prepared for shipment to the diagnostic laboratory for analysis.

### DETAILED DESCRIPTION OF THE PREFERRED EMBODIMENT

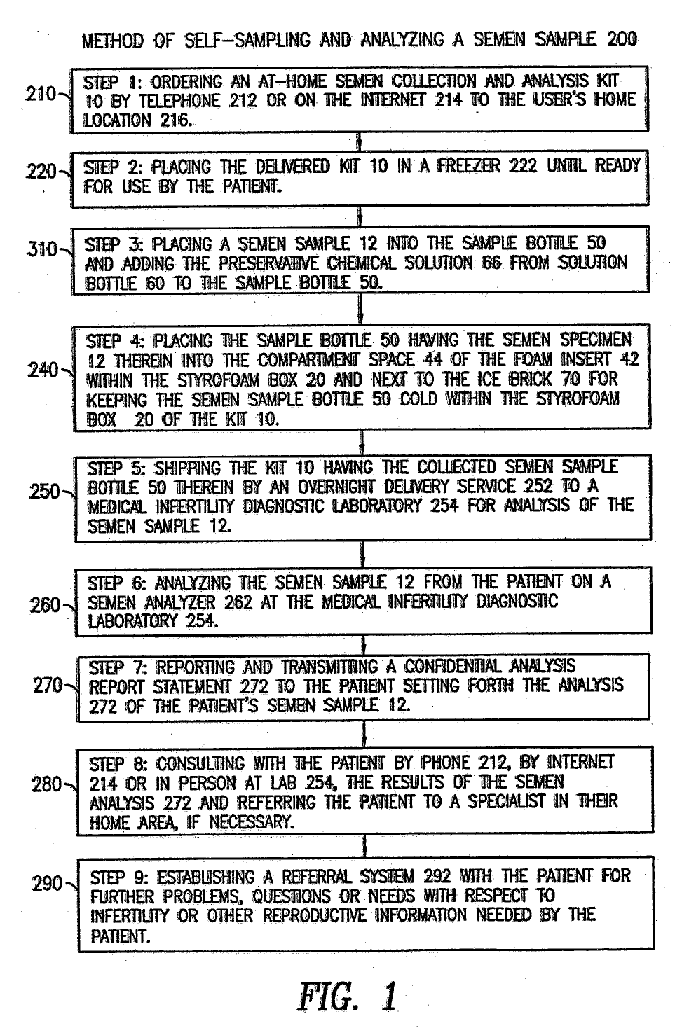

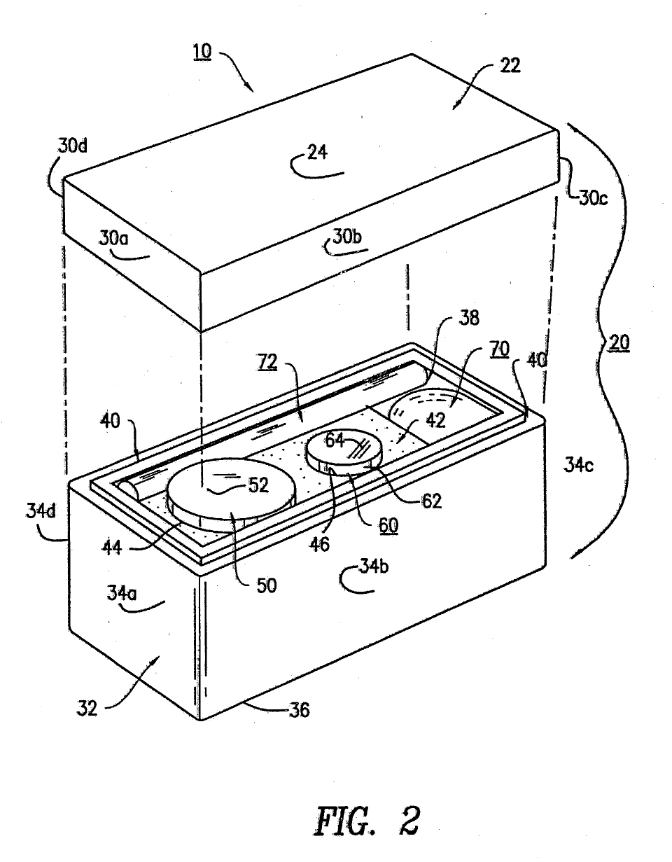

The at-home semen collection and analysis kit 10 and its component parts of the preferred embodiment of the present invention are represented in detail by FIGS. 2 through 5 of the patent drawings. The method 200 of self-sampling and analyzing a semen sample 12 having various procedural and operational steps performed in this medical process is disclosed in the preferred embodiment of the present invention and is represented in detail by FIG. 1 of the patent drawings.

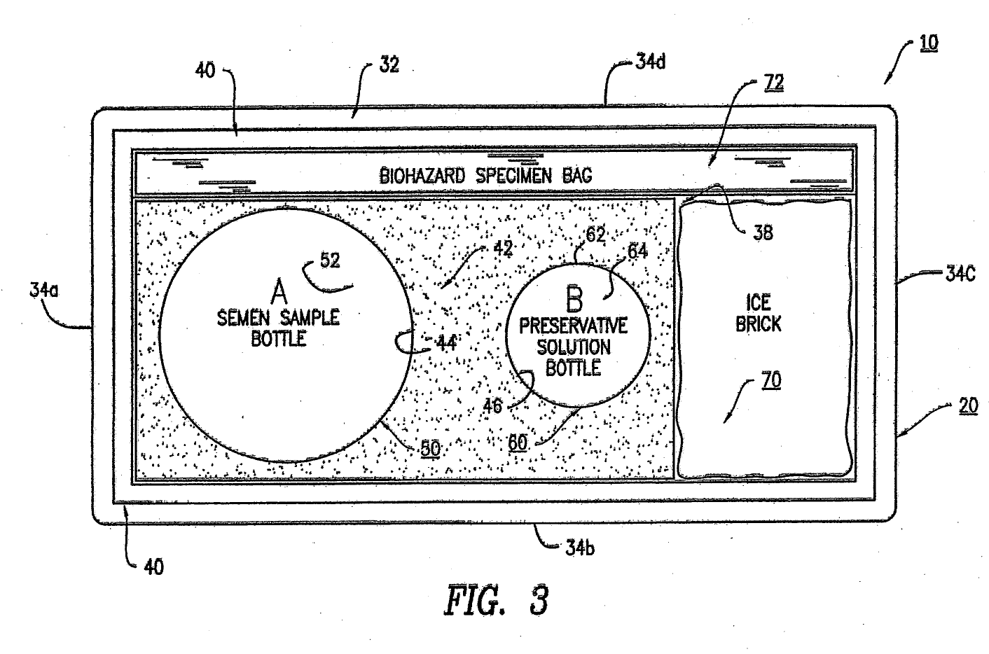

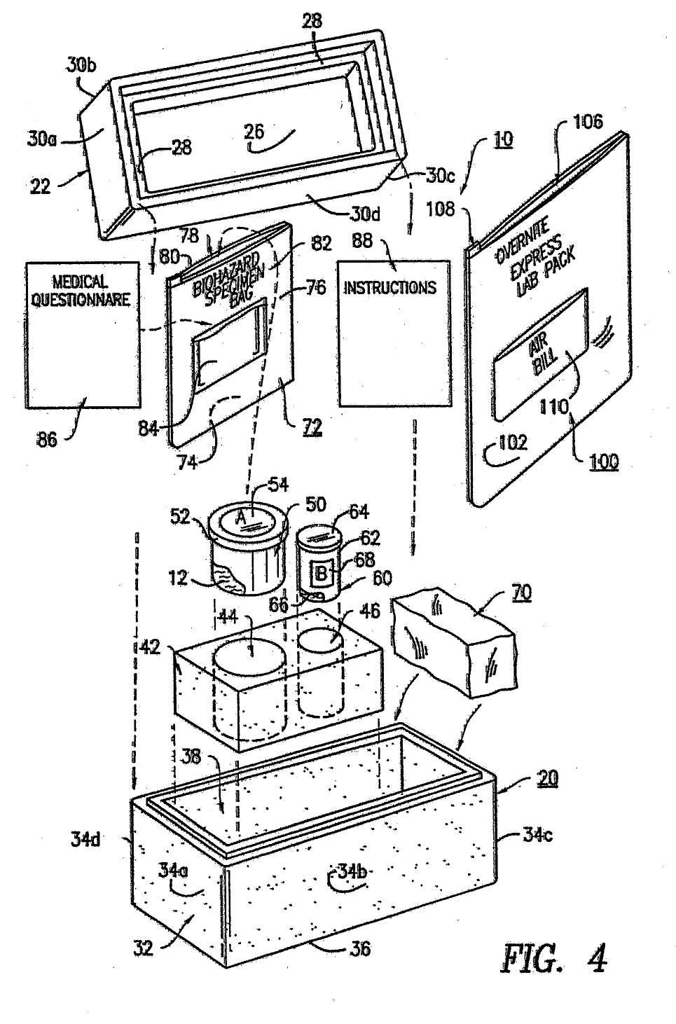

The at-home semen collection and analysis kit 10, as shown in FIGS. 3 and 4, includes a styrofoam box or container 20 having a top cover member 22 and a retaining bottom member 32, a foam insert 42 having a first circular opening 44 for receiving a semen sample collection bottle 50 therein and having a second circular opening 46 for receiving a preservation solution bottle 60 containing a chemical preservative solution 66 therein. Kit 10 further includes a formable, rectangularly-shaped ice brick or pack 70 for keeping the semen sample 12 within the semen sample collection bottle 50 at a cooled temperature in the range of 40° F. to 50° F. when stored in the styrofoam box 20, a biohazard specimen plastic bag 72 having instructional indicia 82 thereon, a patient questionnaire medical response sheet 86, an instruction sheet 88 for giving the method 200 of use for kit 10, a return shipping container 90 and an overnite express lab pack envelope/return mailer 100.

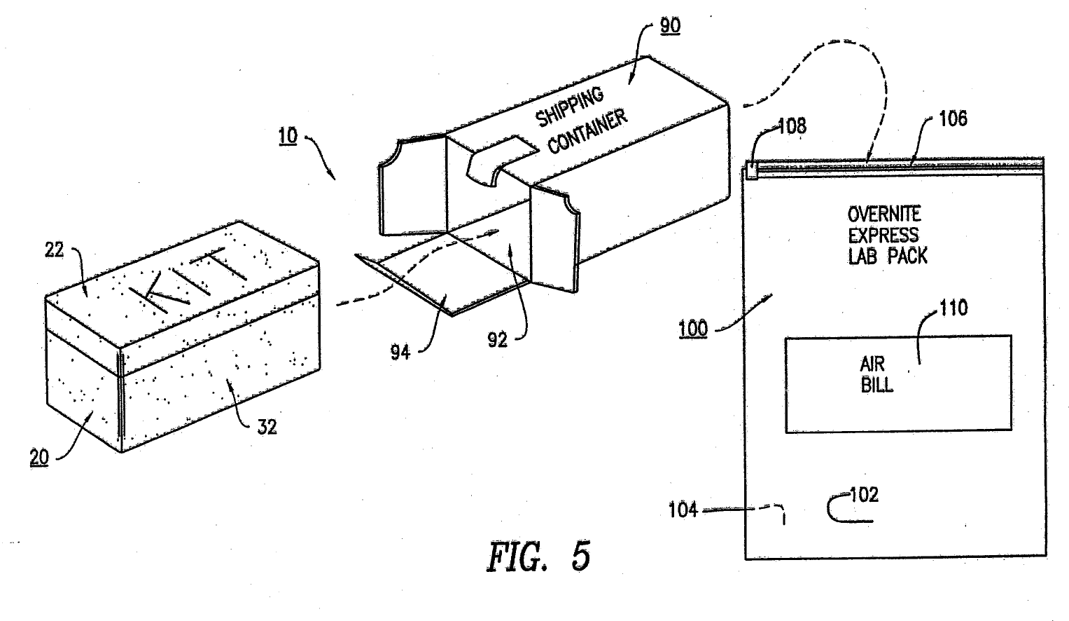

The top cover member 22, as shown in FIGS. 2 and 4, includes an exterior top wall surface 24, an interior top wall surface 26 having interior lap-joint edging 28 and perimeter side walls 30a to 30d. The retaining bottom member 32 includes side walls 34a, 34b, 34c and 34d and a bottom wall 36 for forming an interior compartment 38. Side walls 34a to 34d include perimeter lap joint edging 40 for mating the retaining bottom member 32 with the interior lap-joint edging 28 of the top cover member 22 in order to close the styrofoam box 20 for shipping, as depicted in FIG. 5 of the drawings.

The semen sample collection bottle 50 is made of light-weight plastic materials and includes a plastic screw-on cap 52 having an identification label 54 thereon, as depicted in FIG. 4 of the drawings. The preservation solution bottle 60 is also made of light-weight plastic materials and includes a plastic screw-on cap 62 with a metal seal 64 thereon. The preservation solution bottle 60 contains therein a chemical preservative solution 66 for preserving the semen sample 12 while in transit to the diagnostic laboratory for analysis. The preservation solution bottle 60 also includes an identification label 68 thereon.

The biohazard plastic specimen bag 72, as shown in FIGS. 3 and 4 of the drawings, includes a front wall 74, a rear wall 76 for forming an interior compartment 78 therein and for receiving the semen sample collection bottle (with semen 12) therein, and a zip lock top section 80 for closing the specimen bag 72. The biohazard plastic specimen bag 72 also includes a pocket compartment 84 located on the rear wall 76 for receiving the patient questionnaire medical response sheet 86 therein. Front wall 74 of specimen bag 72 includes instructional indicia 82 thereon for informing the user the use of the specimen bag 72.

The return shipping container 90 is made of light-weight corrugated cardboard material, as shown in FIG. 5, and includes an interior compartment 92 for receiving kit 10 therein and a side cover 94 for enclosing the entire kit 10 therein in preparation for final return shipment to the diagnostic laboratory for analysis.

The overnite express lab pack envelopes/return mailer 100, as shown in FIGS. 4 and 5 of the drawings, includes a front wall 102 and a rear wall 104 for forming an interior compartment 106 therein and for receiving the (packed with kit 10) returner shipping container 90 therein; and a sealant top member 108 for closing the return mailer 100. Front wall 102 includes an attached shipping bill and manifest 110 thereon for shipping the return mailer 100 to a designated diagnostic laboratory for having the semen sample 12 analyzed.

### OPERATION OF THE PRESENT INVENTION

The method 200 and operation of a self-sampling and analyzing kit 10 for analyzing a semen specimen 12 is represented by FIGS. 1, 4 and 5 of the patent drawings. The first step 210, has the user ordering the kit 10 by telephone 212 or on the Internet 214 to the user's place of residence 216 for privacy.

When the user receives the kit 10, as shown in the second step 220, the user places the entire styrofoam box 20 containing the cool pack/ice brick 70 and the preservation solution bottle 60 having a preservative solution 66 therein into the freezer 222. The ice brick 70 is not frozen separately since it will reform into a different shape and then not fit inside the styrofoam box 20.

As shown in the third step 230, when the user is ready to use the kit 10, the user takes out the solution bottle B 60 containing the preservative solution 66 therein from the freezer 222 and places it in warm water for 10 minutes, allowing it to thaw and warm up. The semen sample 12 must be shipped the day it is collected. It is recommended that the user produce the semen sample 12 within 2-3 hours before the pick-up by the overnite delivery service 252. If possible, the user should abstain from ejaculating for 2-3 days before providing the semen sample 12; this will ensure the highest possible sperm count.

The user should not use spermicidal lubricants or any product containing spermicide which would effect the semen sample 12 when being analyzed at the diagnostic lab 254. The user places as much of the semen specimen 12 as possible into the larger plastic bottle 50 labeled A (specimen), trying to get most of the ejaculation into the large bottle A 50, as possible. The user then opens bottle B 60, after it has thawed and warmed in warm water. The user then peels off the metal seal 64 and cap 62 carefully, so as not to cut the user's finger. The user then adds the contents of bottle B 60 (preservative solution 66) to bottle A 50, which now contains the semen sample 12 and screws on cap 52 to seal bottle 50. The user then gently shakes the semen collection bottle A 50 for 10 seconds to ensure adequate mixing. The user then discards the empty bottle B 60 into the user's household garbage.

As shown in the fourth step 240, the semen collection bottle A 50 is readied for shipment. To comply with federal postal regulations, a biohazard specimen bag 72 is provided in the kit 10. After collection of the semen sample 12 in the large bottle labeled A 50, the user then tightly seals bottle A 50 via cap 52, such that the user firmly twists the cap 52 onto bottle A 50 to avoid leakage (in the plane under pressure), which now contains both the user's semen sample 12 and the preservative solution 66. The user also fills out the patient questionnaire response sheet 86 for information needed by the diagnostic lab 254 for proper analysis 272 of the semen sample 12 sent by the user. The patient questionnaire response sheet 86 is folded and placed into the pocket compartment 84 of the biohazard specimen bag 72. The user then now places bottle A 50 inside the biohazard bag 72, seals it via the zip-lock top 80 and places it into the larger opening area 44 of the foam insert 42 inside the bottom retaining member 32 of box 20. After sealing the biohazard specimen bag 72 the user then places it into the foam insert 42 of styrofoam box 20 next to the ice brick 70 for keeping the semen specimen 12 cooled in order to prevent the deterioration of the semen count or motility. The kit 10 is then closed tightly using the top cover member 22, such that the interior lap-joint edging 28 of the top cover member 22 is matched and joined with the perimeter lap-joint edging 40 of the retaining member 32 of styrofoam box 20. Kit 10 is then inserted into the interior compartment 92 of the return shipping container 90 and the side cover 94 is then closed to seal the kit 10 within the return shipping container 90, as shown in FIG. 5 of the drawings.

As shown in the fifth step 250, the user simply follows the shipping instructions sheet 88 sent with the kit 10. The specimen sample 12 must be shipped using an overnite delivery service 252, such as Airborne Express or Federal Express, etc., using the air bill of laden 110 provided by the overnite delivery service 252. The overnite delivery service 252 has already been pre-paid for by the diagnostic lab 254 to ensure the most reliable service. The user calls the overnite delivery service 252 to arrange for a pick-up at the user's home location 216, Monday through Thursday only, shipments are not to be made on Fridays Saturdays or Sundays. The user then places the entire kit 10 and shipping container 90 inside interior compartment 106 of the overnite express lab pack envelope 100 and follows the mailing instructions as provided on the rear wall 104 of the lab pack envelope 108. The user then puts his address on the pre-printed airbill manifest 110, and places the airbill manifest 110 on the front wall 102 of the lab pack envelope 108, where then the user seals the envelope 108 via the sealant top member 108. The user is now ready to call the overnite delivery service 252 for his semen sample/kit 10 pick-up.

As shown in the sixth step 260 of FIG. 1, the semen sample 12 from the user/patient is analyzed by a computer assisted semen analyzer (CASA) 262 at the medical infertility diagnostic laboratory 254. A special microscope reads the semen sample 12 by time-exposure photomicrography and then the results are generated on the computer. This state of the art technology allows each semen specimen 12 to be read exactly in the same manner, eliminating human error or variation in reading the analysis 272 by different lab technicians. The analysis report 272 includes the standard details that any physician would want to know. These include the volume, sperm concentration per milliliter (sperm count) and motility. An accompanying explanation of the meaning of the results will be on the report. This will be sufficient to bring to any health professional for further evaluation and diagnosis. Definitions on the above include: Concentration—normal is over 20 million per milliliter. Motility—at least 50% should be motile. Volume—there should be 2-5 milliliters or cc's of fluid.

As shown in the seventh step 270 of FIG. 1, a confidential analysis report statement 272 is generated via the semen analyzer 262, and this analysis report statement 272 is then reported and transmitted to the patient setting forth the analysis 272 of the patient's semen sample 12. The results are then mailed to the user by first-class mail from the U.S. Post Office within 48 hours of receipt of the semen specimen 12 by the lab 252. The report statement 272 includes information as stated in the previous sixth step 260.

As depicted in the eighth step 280, the user receives the lab analysis report 272 by the United States Postal Service within a few days after the receipt of the semen specimen 12. The user then calls the lab 254 for free consultation to review results of the lab analysis report 272

The final ninth step 290, as shown in FIG. 1 of the drawings, the medical infertility diagnostic laboratory 254 establishes a referral system 292 with the patient for further problems, questions or needs with respect to infertility or other reproductive information needed by the patient.

### ADVANTAGE OF THE PRESENT INVENTION

Accordingly, an advantage of the present invention is that it provides for an at-home semen collection and analysis kit that gives the user an easy way in which to have access to a medical infertility laboratory without the user actually having to go to the laboratory in person.

Another advantage of the present invention is that it provides for an at-home semen collection and analysis kit that includes the necessary collection equipment, the preservative chemicals, an overnite return mailer and box, and instructions for the self-sampling of the user's semen at the privacy of the user's home, whereby the user will receive his confidential report by mail within 48 hours of the semen sample being analyzed at the infertility medical laboratory.

Another advantage of the present invention is that it provides for an at-home semen collection and analysis kit, such that the user is not examined in a cold and impersonal medical facility, as well as having the mental anguish of sharing the user's most intimate medical information of his problems with an unknown medical staff.

Another advantage of the present invention is that it provides for an at-home semen collection and analysis kit that is easy to use, durable in construction, and having simple packing instructions for shipment to the medical infertility laboratory.

Another advantage of the present invention is that it provides for an at-home semen collection and analysis kit that maintains the integrity of the semen sample taken by the user prior to its analysis at the medical infertility laboratory, such that the sperm is in a viable state for proper analysis.

Another advantage of the present invention is that it provides for an at-home semen collection and analysis kit that enables the economic transport of human semen, in a disposable, totally self-contained, passively cooled, inexpensive container that has essentially no moving parts, and that is not equipped with thermostatically controlled cooling arrangements or the like, for periods of time approximating at least 24 to 48 hours.

A further advantage of the present invention is that it provides for an at-home semen collection and analysis kit that can be mass produced in an automated and economical manner and is readily affordable by the user.

A latitude of modification, change, and substitution is intended in the foregoing disclosure, and in some instances, some features of the invention will be employed without corresponding use of other features. Accordingly, it is appropriate that the appended claims be construed broadly and in a manner consistent with the spirit and scope of the invention herein.

What is claimed is:

1. A method of self-sampling and analyzing a semen sample without visiting a doctor's office, utilizing a kit including an insulated box containing an ice brick, a sample bottle, and a preservative solution, comprising the steps of:
a) ordering a kit by telephone or on the Internet;
b) receiving and placing the kit in a freezer until ready for use;
c) placing a semen sample into the sample bottle and adding the preservative solution to said sample bottle;
d) placing the sample bottle into the kit next to the ice brick in the insulated box;
e) shipping the kit and the sample bottle to a diagnostic laboratory for analysis of the semen sample;
f) analyzing the semen sample on a semen analyzer at the diagnostic laboratory; and
g) transmitting a confidential analysis report to the patient setting forth analysis of the semen sample.

2. The method of self-sampling and analyzing a semen sample in accordance with claim 1, further including the step of removing said kit from the freezer.

3. The method of self-sampling and analyzing a semen sample in accordance with claim 1, further including the step of thawing said preservative solution by placing said preservative solution in warm water for 10 minutes to allow said preservative solution to thaw and warm up to room temperature.

4. The method of self-sampling and analyzing a semen sample in accordance with claim 1, further including the step of producing said semen sample within 2 to 3 hours prior to said step of shipping.

5. The method of self-sampling and analyzing a semen sample in accordance with claim 1, wherein said step of placing the semen sample into said sample bottle and adding said preservative solution to said sample bottle includes the step of shaking said semen sample and said preservative solution for 10 seconds to ensure adequate mixing within said sample bottle.

6. The method of self-sampling and analyzing a semen sample in accordance with claim 1, wherein said step of shipping said kit having said semen sample within said sample bottle includes the step of receiving said semen sample at a diagnostic laboratory within 18 hours of said step of shipping.

7. The method of self-sampling and analyzing a semen sample in accordance with claim 1, wherein said step of analyzing said semen sample on a semen analyzer includes the step of testing for sperm volume, sperm count and sperm motility.

8. The method of self-sampling and analyzing a semen sample in accordance with claim 1, wherein said step of transmitting said confidential analysis report of the analysis of said semen samples includes the step of receiving said confidential analysis report within 48 hours after the receipt of said semen sample at the diagnostic laboratory.

9. The method of self-sampling and analyzing a semen sample in accordance with claim 1, further including the step of consulting the patient by phone, by Internet or in person about said confidential analysis report and referring the patient to a specialist in the patient's home area, if necessary.

10. The method of self-sampling and analyzing a semen sample in accordance with claim 1, further including the step of establishing a referral system with the patient for further problems, questions or needs with respect to infertility or other reproductive information needed by the patient.
